(19) United States
(12) Patent Application Publication
(10) Pub. No.: US 2024/0382509 A1
(43) Pub. Date: Nov. 21, 2024

(54) Cannabinoids C- and O-glycosides possessing anti-proliferative and anti-metastatic properties and process for preparation thereof

(71) Applicant: Council of Scientific and Industrial Research, New Delhi (IN)

(72) Inventors: Anindya GOSWAMI, Jammu (IN); Asif ALI, Delhi (IN); Souneek CHAKRABORTY, Jammu (IN); Bashir Khalid MIR, Jammu (IN); Veeranjaneyulu GENNEDI, Taipei (TW); Waseem Iqbal LONE, Jammu (IN); Yedukondalu NALLI, Jammu (IN)

(73) Assignee: Council of Scientific and Industrial Research, New Delhi (IN)

(21) Appl. No.: 18/697,626

(22) PCT Filed: Sep. 27, 2022
(86) PCT No.: PCT/IN2022/050860

§ 371 Date: Apr. 1, 2024

(30) Foreign Application Priority Data
Oct. 1, 2021 (IN) 202111044828

(51) Int. Cl. A61K 31/7048; A61K 31/513; C07H 1/08; C07H 7/06; C07H 15/26

(52) U.S. Cl. CPC A61K 31/7048; A61K 31/513; C07H 1/08; C07H 7/06; C07H 15/26

(57) ABSTRACT

The present invention conveys a fractionation method to isolate different bioactive Phytocannabinoids by solid phase extraction using HP-20 resins and one step method to synthesize C— and O-β-D-glycoside derivatives of Cannabinoid by using a β-D-glucose donor (O-Glycosyl trichloroacetimidate donor) to obtain glycosidic derivatives. The novel C-glycosides of Cannabinoids displayed significant anti-proliferative properties against wide range of human cancer cell lines as compared to their respective parent molecules. Furthermore, the C— and O glycosides of Cannabinoids also showed anti-invasive and anti-migratory activities against metastatic cancer cells of breast and pancreatic origin. When applied in combination with DNA damaging agent (5-Fluorouracil) to colorectal cancer cells, the C— and O-glycosides of Cannabinoids potentiate DNA damaging property and thus augment apoptosis resulting in an increment in the efficacy of DNA damaging drugs.

### FIELD OF THE INVENTION

The present invention relates to C— and O-glycosides of Cannabinoids possessing anti-proliferative and anti-metastatic properties and process for preparation thereof. The present invention relates to the fractionation and separation of distinct class of Cannabinoids by solid-phase extraction using HP-20 resins from Cannabis sativa. The present invention also demonstrates the separation of complex mixtures of Cannabinoids using chemical engineering as a tool for the transformation of a diverse class of molecules. The present invention particularly relates to divergent synthesis of distinct C— and O-glycosidic Cannabinoids with the anti-proliferative and anti-metastatic properties.

### BACKGROUND OF THE INVENTION

Phytocannabinoids and their synthetic derivatives are used as palliative care as a pain reliever, tackling side effects of chemotherapeutic drugs, including nausea and vomiting. They are also implemented as a stimulant to enhance appetite in terminal cancer patients. Although reports exhibiting the role of Phytocannabinoids as anti-tumorigenic agents are there, still, there prevails a major scope in the development of Phytocannabinoids from a mere supporting therapeutic agent to a potential anticancer drug [Hermanson et al. 2011 Cancer Metastasis Rev. Dec; 30 (3-4): 599-612 & Blake et al. 2017, Ann PalliatMed; 6(Suppl 2): S215-S222]. The Phytocannabinoids interacts with the receptors of the Endocannabinoid system viz. CB₁ & CB₂ (G-protein receptors). Although these receptors are mainly located in the central nervous system and peripheral tissues, recent studies suggest their distinct pro-proliferative roles in malignant tumor tissues. Phytocannabinoids belongs to the group of C₂₁ terpenophenolic compounds primarily obtained from plants of genus Cannabis and consists of the seven most abundant derivatives of Cannabinoids viz THC, CBD, Cannabichroene (CBC), Cannabidiolic acid (CBDA), Δ⁸-THC, Cannabigerol (CBG) and Cannabidivarin (CBDV) [Daris et al. 2019, Bosn J Basic Med Sci;19(1):14-23]. Rationally, efforts unleash to develop a new method for the isolation of diverse compounds from an enriched fraction of Phytocannabinoids and they are designated as Δ⁹-tetrahydrocannabivarin 1 (THCV), Δ⁹-tetrahydrocannabinol 2 (Δ⁹-THC), Δ⁸-tetrahydrocannabinol 3 (Δ⁸-THC), Cannabinol 4 (CBN), Cannabidiol 5 (CBD) [Ali et al. 2018, Tet. Lett. 59 (25), 2470-2472& Ali et. al. 2019, Bio. org. Med. Chem. Lett. 29, 1043-46].

Cancer metastasis is the most vital hallmark of cancer that accounts for the majority of cancer-related mortality, and tumor relapse [Hanahan et al. 2011, Cell, Vol 144, Issue 5,646-674].Δ⁹-Tetrahydrocannabinol (2) is the earliest derivative of Phytocannabinoids moderately explored for its anticancer activities but its role in cancer cells invasion and metastasis warrants more in-depth studies [Ganju et al. 2008, Oncogene 27, 339-346]. DNA damaging drugs are widely used in clinical practice, but most of them consequently develop resistance to therapies down the course of treatment. Burgeoning pieces of evidence elicit that cancer cells upon treatment with these DNA damaging drugs induce EMT during the initial phase of treatment, leading to restraining chemo-sensitivity [Chakraborty et al. 2019, Cell Death and Disease, 10:467 & Fischer et al. 2015, Nature, 527(7579), 472-476]. To tackle this issue, rationally, anti-metastatic molecules can be implemented in combination with DNA damaging agents. Recently, anti-EMT molecules viz. curcumin, mocetinostat, and metformin are used in combination with 5-Fluorouracil, doxorubicin, and gemcitabine to overcome drug resistance by these DNA damaging agents [Chakraborty et al. 2020, Cancer Metastasis Rev, Vol 39, 553]. The patent application PCT/IN2018/050060 discloses Indolyl kojyl methane analogue IKM5 as an anti-metastatic molecule that augmented the efficacy of doxorubicin in GRP-78 mediated EMT induction.

The present invention describes the fractionation method for Cannabinoids by solid-phase extraction employing HP-20 resins. Furthermore, an enriched fraction was glycosylated, leading to the synthesis of several new C— and O-Cannabinoid-β-D-glycosides. All these compounds have largely improved solubility in aqueous solutions. This increased aqueous solubility enables novel oral delivery options for Cannabinoids, as well as targeted delivery and release of Cannabinoids within the intestines through glycoside prodrug metabolism [Watanabe et al., 2007 Forensic Toxicol. 25(1), 16-21]. Glycosides are known to trigger direct therapeutic effects; it improves drug bioavailability and drug pharmacokinetics, including more site specific or tissue specific manner that helps drug delivery in a more consistent way in plasma and sustained delayed release of the molecule [Friend et al., 1984. J Med Chem. 27, 261-266]. In a nutshell, C— & O-β-D-glycosidic Cannabinoids may play an important role in bypassing the digestive tract and colon, such as intravenous delivery, that will enable targeted delivery to other cells and tissues [Friend et al., 1985. J Med Chem. 28, 51-57].

### OBJECTIVE OF THE INVENTION

The main objective of the present invention is to provide C— and O-glycosides of Cannabinoids. Another objective of the present invention is to develop a new method for fractionation and separation of distinct Cannabinoids with the aim of executing chemical diversification to prepare novel C— and O-glycosides of Phytocannabinoids. Another objective of the present invention is to provide a process for preparation of C— and O-glycosides of Cannabinoids. Yet another objective of the present invention is to provide C— and O-glycosides of Cannabinoids possessing anti-proliferative and anti-metastatic properties.

### SUMMARY OF THE INVENTION

The present invention describes the fractionation method for Cannabinoids (1-5) by solid-phase extraction employing HP-20 resins. Furthermore, an enriched fraction (1-5) was glycosylated, leading to the synthesis of new C— and O-Cannabinoid-β-D-glycosides.

An aspect of the present invention provides a Cannabinoid C— and O-glycoside compound having the formula (A),

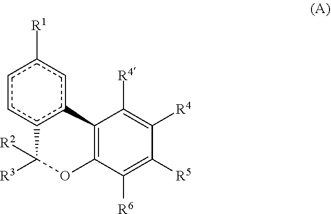

Wherein,
R¹, R² and R³ are each independently selected from the group consisting of H, OH, alkyl, alkenyl and alkynyl;
R⁴ and R^{4′} are each independently selected from the group consisting of H, O-glycoside, substituted-O-glycoside, C-glycoside, substituted-C-glycoside, —(CH₂)ₙ—O-glycoside and (CH₂)ₙ—C-glycoside;
R⁵ and R⁶ are each independently selected from the group consisting of H, halogen,—CN,—NO₂, —OH, alkyl, —O-alkyl and —COOH.

Another aspect of the present invention provides the Cannabinoid C— and O-glycoside compound is selected from the group consisting of

| | |
|---|---|
| 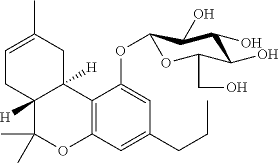 Δ⁸-tetrahydrocannabivarin-1-O-β-D- glucopyranoside (THCOG) 1a | 1a |
| 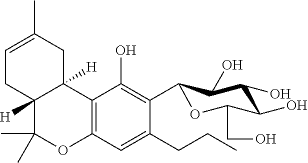 Δ⁸-tetrahydrocannabivarin-2-C-β-D- glucopyranoside (THCCG) 1b | 1b |
| 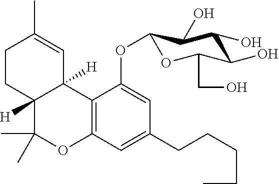 Δ⁹-tetrahydrocannabinol-1-O-β-D- glucopyranoside (9-THCOG) 2a | 2a |
| 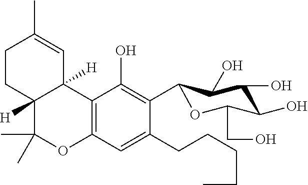 Δ⁹-tetrahydrocannabinol-2-C-β-D- glucopyranoside (9-THCCG) 2b | 2b |
| 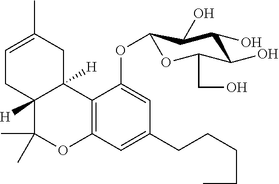 Δ⁸-tetrahydrocannabinol-1-O-β-D- glucopyranoside (8-THCOG) 3a | 3a |
| 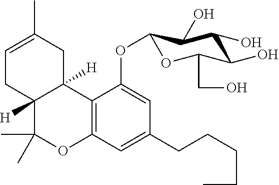 Δ⁸-tetrahydrocannabinol-2-C-β-D- glucopyranoside (8-THCCG) 3b | 3b |
| 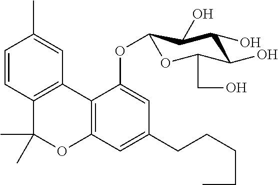 Cannabinol-1-O-β-D-glucopyranoside (CBNOG) 4a | 4a |
| 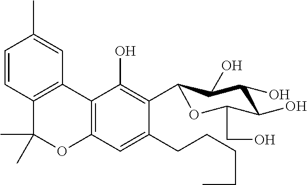 Cannabinol-2-C-β-D-glucopyranoside (CBNCG) 4b | 4b |

Yet another aspect of the present invention provides that the Cannabinoid C— and O-glycoside compounds possess anti-proliferative and anti-metastatic properties and effectively abrogates proliferation of different cancer cells in-vitro.

Another aspect of the present invention also provides a process for the synthesis of the Cannabinoid C— and O-glycoside compound having the formula (A),

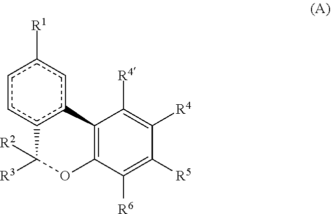

Wherein,
R¹, R² and R³ are each independently selected from the group consisting of H, OH, alkyl, alkenyl and alkynyl;
R⁴ and R^{4′} are each independently selected from the group consisting of H, O-glycoside, substituted-O-glycoside, C-glycoside, substituted-C-glycoside, —(CH₂)ₙ—O-glycoside and (CH₂)ₙ—C-glycoside;
R⁵ and R⁶ are each independently selected from the group consisting of H, halogen, —CN, —NO₂, —OH, alkyl, —O-alkyl and —COOH, comprising the steps of
a. extracting the ground leaves of Cannabis sativa with hexane at room temperature to yield the crude extract,
b. subjecting the extract to fractionation in an open column using the HP-20 as a solid phase and a gradient solvent system with H₂O—MeOH of 9:1, 8:2, 7:3, 6:4, 1:1, 4:6, 3:7, 2:8, 1:9 and 100% MeOH, resulting in Cannabinoid enriched fractions (1 to 10).
c. co-evaporating trichloroacetimidate glycoside donor (I)

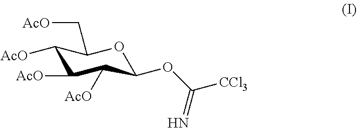

wherein R is H and Ac and Cannabinoid enriched fractions to obtain Cannabinoid C— and O-glycoside compound having the formula (A).

An aspect of the present invention provides the Cannabinoid enriched fraction co-evaporating with trichloroacetimidate glycoside donor (I) is fraction 5.

Another aspect of the present invention provides a pharmaceutical composition comprising Cannabinoid C— and O-glycoside compound having the formula (A) along with pharmaceutically acceptable excipients.

Another aspect of the present invention provides a pharmaceutical composition comprising Cannabinoid C— and O-glycoside compound having the formula (A), DNA damaging agent 5-Fluorouracil and pharmaceutically acceptable excipients.

Another aspect of the present invention provides that the pharmaceutically acceptable excipients are selected from a group consisting of inert diluents selected from calcium carbonate, sodium carbonate, lactose, calcium phosphate or sodium phosphate; oily suspensions; sweetening agents selected from glycerol, propylene glycol, sorbitol and sucrose.

### BRIEF DESCRIPTION OF THE DRAWINGS

FIG. 1 illustrates the process steps for fractionation and generation of C— and O-β-D-glycoside of Cannabinoids.

FIG. 2 shows anti-proliferative property of the C— and O-glycosides of Cannabinoids.

FIG. 3 illustrates anti-migratory property of Cannabinoid derivatives in MIAPaCa-2.

FIG. 4 illustrates inhibition of wound closure by Cannabinoid derivatives in MIAPaCa-2 cells.

FIG. 5 illustrates anti invasive property of compound 2a in pancreatic cancer cell.

FIG. 6 illustrates compound 2a abrogates invasion and metastasis of highly aggressive breast cancer cells.

FIG. 7 illustrates compound 2a stalls tumor growth and metastasis in 4T1 mouse metastatic model.

FIG. 8 illustrates compound 2a in combination elevates the DNA damaging potential of 5FU in colon cancer.

FIG. 9 illustrates compound 2a in combination increases the apoptosis potential of 5FU in 1×10⁶ HCT-116 cells.

FIG. 10 illustrates the divergent synthesis of C— and O-glycosides of Cannabinoids.

### DETAILED DESCRIPTION OF THE INVENTION

The invention will now be described in detail in connection with certain preferred and optional embodiments, so that various aspects thereof may be more fully understood and appreciated.

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

### Definitions

The term Divergent synthesis used herein refers to chemical engineering a tool for structural modification of natural metabolite.
Cannabinoids: Cannabinoids used herein refers to the class compounds found in Cannabis.
Fractionation: Fractionation used herein refers to a separation process in which a certain quantity of a mixture makes it possible to isolate more than two components in a mixture in a single run.
Aqueous solubility: Aqueous solubility used herein refers to a key physicochemical process required to characterize an active pharmaceutical ingredient (API) during drug discovery and beyond. It also plays a significant role in formulation selection and subsequent development processes.
Oral pharmaceutical delivery (OPD): used herein refers to the most preferred drug administration route due to convenience, cost-effectiveness, which includes aqueous solubility, membrane permeability, and chemical and enzymatic stability of drugs.
Targeted delivery (TD): used herein refers to the method for delivering medication to a patient to increase the concentration of the medication in some parts of the body relative to others.
Bioavailability: used herein refers to the fraction of the dose administered drug that reaches the systemic circulation unchanged.
Base catalysis: used as a chemical reaction, which catalysed by a base and the proton acceptor
Anomeric center (AC): used herein refers to a stereocentre created from the intramolecular formation of an acetal (or ketal) of a sugar hydroxyl group and an aldehyde (or ketone) group.
The term cytotoxicity used herein is an in vitro test to study the toxicity caused by any chemotherapeutic agent.
The term Epithelial to mesenchymal transitions (EMT): used herein refers to a phase wherein adenocarcinoma cells of epithelial origin changes its structure to cells of mesenchymal origin that facilitate the cells to attain migratory property for further metastasis.
The term podial structures (invadopodia/filopodia): used herein describes the phase of cellular transformation wherein the mesenchymal cells display membranous protrusions towards the migratory front which is indicated as lamellipodia and filopodia which aid in cellular movement.
The word metastatic nodules: used herein refer to distant migration of cancer cells from primary tumor site to a distant site forming a random patches pattern.
The term DNA damage: used herein refers to a situation where the DNA within the cell is altered by the presence of agents which either break the backbone of the double helix or react with the bases to form chemical intermediates or get substituted in place of natural bases resulting in stalling of the replication fork and subsequent firing of signals related to DNA repair and in extreme cases apoptosis.
The term chromosomal instability (CIN): used herein refers to a form of genomic instability that results in alteration of chromosomal structure, ranging from either breakage of chromosomal part, centromeric loss, and chromosomal fusion to complete deletion.
The term DNA repair refers to the primary cellular response to DNA damage, eventually recruiting several DNA binding proteins that help in correcting the various types of damages resulting in the reversal of DNA damage responses.
Combinatorial treatment means incorporating more than one therapeutic agent to treat a specific disease condition. The co-administration of active therapeutic agents may be simultaneous or periodic at a fixed ratio of the active ingredients.
The term drug efficacy used herein refers to the DNA damaging agent's property to cause a substantial amount of damage in the DNA molecule that ultimately escalates as programmed cell death.
The term drug potentiation used herein refers to the synergism of two or more drugs in combination shows elevated therapeutic effect compared to drugs administered individually.
The term wound healing used herein refers to the capacity of cancer cells to replenish any wound created by external agents by activating cellular migration.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. The present disclosure is not to be limited in scope by the specific embodiments described herein, which are intended for the purposes of exemplification only. Functionally-equivalent products, compositions, and methods are clearly within the scope of the disclosure, as described herein.

The present invention describes the solid-phase extraction using HP-20 resins a classical method of fractionation of distinct Cannabinoids and a one-step method to produce distinct C— and O-glycosidic Cannabinoids that possess anti-proliferative activity against wide range of cancers.

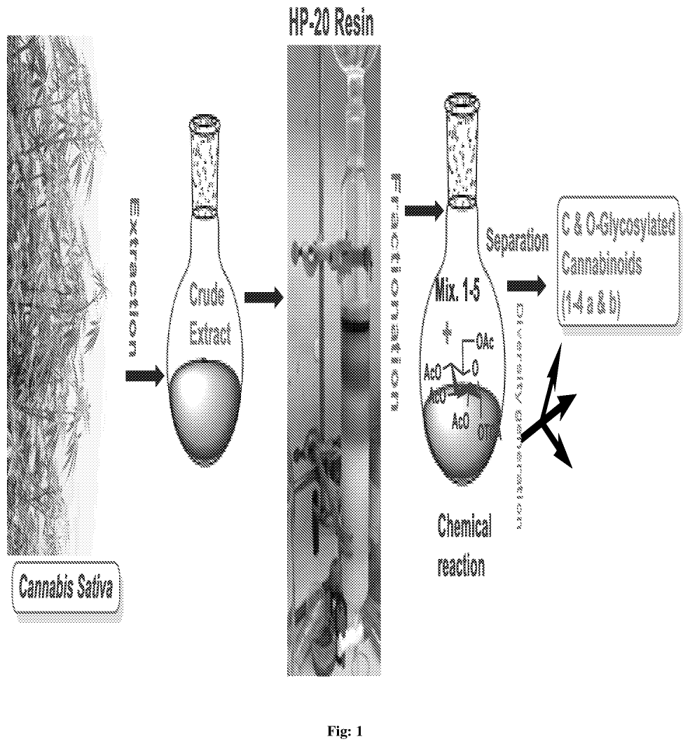

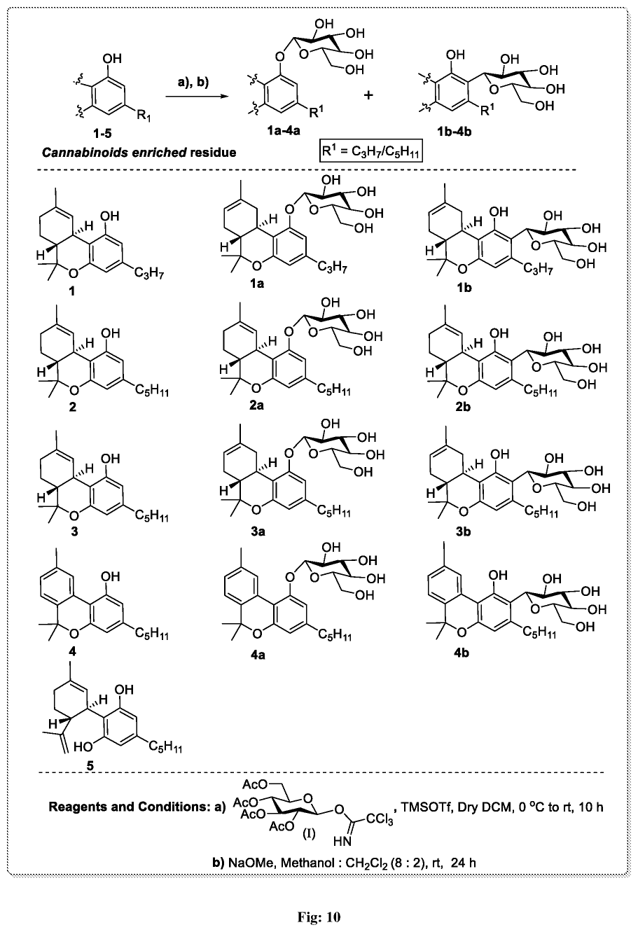

In an embodiment of the present disclosure, there is provided a process for the fractionation and separation of distinct class of Cannabinoids. The process demonstrates that fractionation and separation of distinct class of Cannabinoids by solid-phase extraction using HP-20 resins could be utilized as an alternative source for diverse molecules with interesting pharmacological activities. FIG. 1 provides the process for fractionation and generation of C— and O-β-D-glycoside of Cannabinoids. The ground leaves of Cannabis sativa was extracted with hexane at room temperature to yield the crude extract. The extract was subjected to fractionation in an open column using the HP-20 as a solid phase and different gradient solvent system with H₂O—MeOH resulting in fractions 1-10. Wherein fraction 5, a specific fraction, is the mixture of compounds (1-5) of FIG. 10:

Furthermore, this invention discloses the anti-proliferative and anti-metastatic potential of active O-glycosidic of Δ⁹-tetrahydrocannabinol (compound 2a) obtained by chemical diversification of C— and O β-D-glycoside of Cannabinoids extracted from Cannabis sativa. The present invention describes the chemical process underscoring the transformation of a wide range of synthetic C— and O-glycosides of Cannabinoids in one step. The present invention described the synthesis of the O-glycosyl trichloroacetimidate donor (I) from 1-O-unprotected β-D-glucose-2,3,4,6-tetraacetate and trichloroacetonitrile under base catalysis [Ali. A et al., 2010, Tetrahedron 66, 4357-4369].

Herein described is the reaction with different positions of alcohols in Cannabinoids in the presence of acid catalyst which afforded distinct C— and O-β-D-glycoside by inversion of configuration at the anomeric center.

The β-configurations of compounds (1-4a&b) C— and O-glycosides were authenticated by the appearance of the anomeric protons as doublets at δ 4.93, 4.92, 4.96, 5.06 (1-4a) with the coupling constant values of J _{1,2} 7.1, 7.2, 7.4, and 7.7 Hz, respectively whereas compounds (1-4b) anomeric protons as doublets at δ4.66, 4.52, 4.54, 4.63 with coupling constant values of J _{1,2} 9.7, 9.8, 9.7, and 9.7 Hz.

The existence of C-glycosides in nature is generally found to be limited among mammalian systems, but such derivatives are widely distributed in plants and endophytic microbes [Gaoni, Y. et al., 1971, J. Am. Chem. Soc. 93, 217]. Several synthetic methods were developed to synthesize C-glucosides of Cannabinoids [Yagen, B., et al., 1977, J. Am. Chem. Soc. 99, 6444; Zehavi, U. et al., 1981, Carbo. Res 96, 1].

In the present invention, trimethylsilyl trifluoromethanesulfonate (TMSOTf), 0.89 mmol is introduced as a condensing reagent in the reaction mixture of a fractionated Cannabis extract, which is the mixture of compounds 1-5, and O-glycosyl trichloroacetimidate donor (I) which afforded a mixture of compounds (1a-4a and 1b-4b), wherein the C— and O-glycoside is in a ratio of 6:4 as a viscous material (FIG. 1 and FIG. 10). All the compounds prepared in this reaction were identified by comparison with its spectroscopic data (NMR, Mass).

The C— and O-glycosides of Cannabinoids of interest having anti-proliferative activity against wide range of cancers, produced from the process are represented by the compound of formula (A),

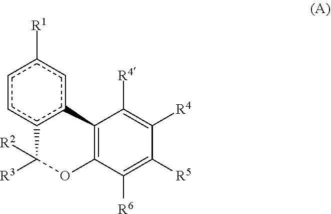

Wherein,
R¹, R² and R³ are each independently selected from the group consisting of H, OH, alkyl, alkenyl and alkynyl;
R⁴ and R^{4′} are each independently selected from the group consisting of H, O-glycoside, substituted-O-glycoside, C-glycoside, substituted-C-glycoside, —(CH₂)ₙ—O-glycoside and (CH₂)ₙ—C-glycoside;
R⁵ and R⁶ are each independently selected from the group consisting of H, halogen, —CN, —NO₂, —OH, alkyl, —O-alkyl and —COOH.

An embodiment of the present invention provides the pharmaceutical compositions comprising Cannabinoid C— and O-glycoside compound having the formula (A) along with pharmaceutically acceptable excipients. Cannabinoids therapeutic potential pursued as new treatment options in diverse medical fields such as neurology, oncology, gastroenterology and pain management. Due to extreme hydrophobicity and instability of Cannabinoids compounds, and as a result, formulation and delivery options are severely limited. Chemically glycosylation strategy to alter the physicochemical properties of small molecules, often improving their stability and aqueous solubility, as well as enabling site-specific drug targeting strategies.

The present invention elaborates the synthesis of distinct C— and O-Cannabinoid glycoside prodrug useful as pharmaceutical agents without glucose cleavage, where they exhibit novel pharmacodynamic properties compared to the parent compound alone. The increased aqueous solubility of the Cannabinoid glycoside prodrugs of the present invention also enables new formulations for delivery in transdermal or aqueous formulations that would not have been achievable if formulating hydrophobic Cannabinoid molecules. The formulations of C— & O-Cannabinoid glycosides provide a method for facilitating the transport of a Cannabinoid drug to the brain through intranasal, stereotactic, or intrathecal delivery, or delivery across the blood brain barrier of a subject comprising administering a Cannabinoid glycoside prodrug in accordance with the present invention to a subject in need thereof.

The pharmaceutical compositions are formulated for oral administration which can be formulated, for example, as tablets, troches, lozenges, aqueous or oily suspensions, dispersible powders or granules, emulsion hard or soft capsules, or syrups or elixirs. The pharmaceutical compositions can be prepared as per known standard methods and may be used by any agents selected from the group of sweetening agents, flavouring agents, colouring agents and preserving agents in order to provide pharmaceutically elegant and palatable preparations. For tablets formulation require the active ingredient in admixture with suitable non-toxic pharmaceutically acceptable excipients including, for example, inert diluents, such as calcium carbonate, sodium carbonate, lactose, calcium phosphate or sodium phosphate; granulating and disintegrating agents, such as corn starch, or alginic acid; binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period. For example, a time delay material such as glyceryl monosterate or glyceryl distearate may be employed to further facilitate delivery of the drug compound to the desired location in the digestive tract. Pharmaceutical compositions for oral use can also be prepared as hard gelatin capsules as active ingredient and mixed with an inert solid diluent, for example, calcium carbonate, calcium phosphate or kaolin, or as soft gelatine capsules wherein the active ingredient is mixed with water or an oil medium such as peanut oil, liquid paraffin or olive oil. Pharmaceutical compositions can be formulated as oily suspensions by suspending the active compound(s) in a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol. Sweetening agents such as those set forth above, and/or flavouring agents may be added to provide palatable oral preparations. These compositions can be preserved by the addition of an anti-oxidant such as ascorbic acid. The pharmaceutical compositions s can be formulated as a dispersible powder or granules, which can subsequently be used to prepare an aqueous suspension by the addition of water. Such dispersible powders or granules provide the active ingredient in admixture with one or more dispersing or wetting agents, suspending agents and/or preservatives. Suitable dispersing or wetting agents and suspending agents are exemplified by those already mentioned above. Additional excipients, for example, sweetening, flavouring and colouring agents, can also be included in these compositions.

Pharmaceutical compositions can be formulated as a syrup or elixir by combining the active ingredient(s) with one or more sweetening agents, for example glycerol, propylene glycol, sorbitol or sucrose. Such formulations can also optionally contain one or more demulcents, preservatives, flavouring agents and/or colouring agents.

### EXAMPLES

Following are the examples given to further illustrate the invention and should not be construed to limit the scope of the present invention.

### Example 1 Extraction and Fractionation

The leaves of Cannabis sativa, were collected from the Botanical garden of the CSIR-Indian Institute of Integrative Medicine, Jammu (India). The plant along with a voucher specimen (IIIM 23453) was deposited at the Herbarium of the IIIM, Jammu (India). The isolation and synthetic modification of Cannabinoids from the leaves of Cannabis sativa, collected from J&K region is being disclosed. A ground leaves of Cannabis sativa (5.0 Kg) was extracted with hexane at room temperature to yield 500 g of crude extract. The extract was subjected to fractionation in an open column using the HP-20 as a solid phase and a gradient solvent system with H₂O—MeOH of 9:1, 8:2, 7:3, 6:4, 1:1, 4:6, 3:7, 2:8, 1:9 and 100% MeOH, resulting in ten fraction (Fr. 1-10). The fraction Fr. 5 (40 g), eluted (at 70% MeOH in H₂O) was the mixture of compounds (1-5) (FIG. 1 and FIG. 10) authenticated by HPLC. Furthermore, the Fr. 5 (1 g) was dissolved in HPLC-grade acetonitrile (10 mL) followed by filtration through a 0.45 μm filter; the resulting solution was subjected to preparative HPLC. As a result, five compounds were purified. The pre-parative HPLC used was equipped with Agilent 1260 series with PDA detector. The elution performed with water (A) and acetonitrile (B) as mobile phase (0-35 min. 19% B; 35-45 min, 19-40% B; 45-50 min, 40% B; 50-57 min, 40-95% B; 57-65 min, 95% B; 65-70 min, 95-19% B; 70-75 min, 19% B). Eclipse XDB-C-18 (5 μm, 250 ×20 mm) column was used at a flow rate of 1.5 mL/min (run time 45 min; column temp. 30° C.; injection vol. 100 μL; UV detection at 215 nm). The purity of the compounds was >95% by HPLC/UV analysis. Purified compounds were identified and characterized by IR, UV, HRESIMS, NMR (1D and 2D spectroscopy), and their comparison with those of the literature data.

### Example 2 Synthesis of O-(2,3,4,6-tetra-O-acetyl-α-D-glucopyranosyl)trichloroacetimidate (I)

To a suspension of D-glucose (6.3 g. 35.00 mmol) in 100 mL of pyridine, 60 mL of acetic anhydride and a catalytic amount of DMAP were added at 4° C. The reaction mixture was allowed to warm at room temp. and stirred for 5 h. The TLC analysis indicated the complete conversion of starting material into products. Furthermore, copper sulphate solution was added and stirred for 30 minutes. Finally, the reaction mixture was transferred to a separating funnel and extracted with ethyl acetate (2 ×200 mL), and the combined organic layers were washed with brine H₂O (3 times). Finally, the organic layer was dried over Na₂SO₄, filtered, and concentrated in a vacuum. The trace amount of pyridine was removed by co-evaporating with toluene, and finally (white solid) was obtained in quantitative yield. To a solution of crude β-D-glucose-1,2,3,4,6-pentaacetate (12.4 g, 32.00 mmol) in DMF (180 mL) was added ammonium acetate (NH₄OAc) (5.7 g. 7.5 mmol) and stirring overnight. Brine water (200 mL) was added and extracted with ethyl acetate (EtOAc) (3×100 mL); the combined organic phases were dried (Na₂SO₄). The solvent was removed under reduced pressure to give faintly orange viscous oil. Furthermore, the crude residue was subjected to flash column chromatography using 40% EtOAc/hexane to afford 9.8 g product as (gummy solid) in 89% yield.

The β-D-glucose-2,3,4,6-tetraacetate (9.0 g. 25.90 mmol) was dissolved in 200 mL anhydrous CH₂Cl₂ and added K₂CO₃ (8.90 g. 64.4 mmol), allowed to stir at room temperature for 5 minutes. Furthermore, CCl₃CN (3.16 mL, 32.00 mmol) was added to the reaction mixture and stirred for 3 h. The TLC analysis indicates the complete conversion of starting material, filtered through celite to remove K₂CO₃ and concentrated in vacuum. Finally, the crude reaction mixture was purified through column chromatography by using 20-40% EtOAc/hexane to afford 11.6 g product as white waxy material in 91% of yield.

### Example 3 Glycosylation of Cannabinoids Enriched Extract

The trichloroacetimidate glycoside donor (I) (5.5 g. 11.25 mmol) and Cannabinoids enriched residue (1.5 g) were co-evaporated with anhydrous toluene, dried for 2 h on high vacuum, and then dissolved in dry CH₂Cl₂ (100 mL) and freshly activated 4 g of 4 Å molecular sieves were added. The suspension was then stirred under nitrogen at room temperature for 30 min. The reaction mixture was cooled to 0° C. (Immersion cooler) and treated with TMSOTf solution (200 μL, 0.89 mmol) and then warm to room temperature. After stirring for 10 h, the reaction was neutralized with trimethylamine followed by filtration through celite to remove molecular sieves, concentrated in vacuum, the crude material was dissolved in ethyl acetate and washed with a saturated solution of NaHCO₃ followed by H₂O. Finally, extracted with ethyl acetate (EtOAc) (3×200 mL), the combined organic phases were dried (Na₂SO₄) and concentrated in a vacuum. The crude material was dissolved in MeOH/CH₂Cl₂ (8/2, 200 mL) and treated with NaOMe (2.0 g, 37.01 mmol). The reaction mixture was allowed to stir at rt for 24 h. The reaction was neutralized with IR 120 H⁺resin and filtered through the celite, the filtrate was concentrated, and the crude residue was subjected to column chromatography by using MeOH/CHCl₃ (10%) to afford the Cannabinoid-glycosides as a mixture. The Cannabinoid-glycosides mixture was dissolved in MeOH and subjected to Semi-Prep-HPLC, and the following glycosides were isolated. The NMR spetra reveals that compound 1 is transformed into 1a as major and 1b was minor product.

### Δ⁸-Tetrahydrocannabivarin-1-O-β-D-Glucopyranoside (8-THCVOG) (1a)

¹H NMR (400 MHz, MeOD) δ 6.55 (d, J=0.9 Hz, 1H), 6.30 (s, 1H), 5.44 (s, 1H), 4.93 (d, J=7.1 Hz, 1H), 4.01−3.90 (m, 1H), 3.75−(dd, J=12.0, 5.3 Hz, 1H), 3.55−3.50 (m, 1H), 3.49−3.46 (m, 1H), 3.47−3.43 (m, 1H), 3.44−3.41 (m, 1H), 2.86 (td, J=11.1, 4.7 Hz, 1H), 2.57−2.44 (m, 2H), 2.12−2.10 (m, 1H), 1.85 (dd, J=21.5, 7.3 Hz, 1H), 1.75 (dd, J=11.5, 4.1 Hz, 2H), 1.71 (s, 3H), 1.68−1.591 (dd, J=15.1, 7.3 Hz, 2H), 1.36 (s, 3H), 1.32 (d, J=10.2 Hz, 1H), 1.09 (s, 3H), 0.96 (t, J=7.3 Hz, 3H); ¹³C NMR (100 MHz, MeOD) δ 156.81, 154.05, 142.01, 134.57, 119.01, 112.92, 111.17, 106.38, 100.40, 77.15, 76.75, 76.18, 73.82, 70.15, 61.29, 45.50, 37.62, 36.61, 31.66, 27.60, 26.62, 23.99, 22.35, 17.26, 12.79; (+) HRESIMS m/z 449.2510 [M+H]⁺ (calcd for C₂₅H₃₇O₇ 449.2534).

### Δ⁸-Tetrahydrocannabivarin-2-C-β-D-Glucopyranoside (8-THCVCG) (1b):

¹H NMR (400 MHz, MeOD) δ 6.19 (s, 1H), 5.45 (s, 1H), 4.66 (d, J=9.7 Hz, 1H), 3.92 (dd, J=12.1, 2.3 Hz, 1H), 3.85 (dd, J=12.2, 4.5 Hz, 1H), 3.72 (t, J=9.2 Hz, 1H), 3.61−3.53 (m, 1H), 3.50 (d, J=8.9 Hz, 1H), 3.48−3.44 (m, 1H), 3.44−3.44 (m, 1H), 2.92−2.78 (m, 1H), 2.68 (td, J=11.1, 4.6 Hz, 1H), 2.46−2.29 (m, 1H), 2.23−2.11 (m, 1H), 1.92−1.80 (m, 1H), 1.78−1.67 (m, 5H), 1.66−1.52 (m, 2H), 1.37 (s, 3H), 1.07 (s, 3H), 0.99 (t, J=7.3 Hz, 3H); ¹³C NMR (100 MHz, MeOD) δ 155.56, 154.11, 140.86, 134.53 (2xC), 118.93, 112.08, 109.85, 81.25, 79.16, 78.29, 76.08. 72.40, 69.74, 60.70, 45.48, 35.87, 35.09, 31.92, 27.66, 26.60, 23.73, 22.34, 17.36, 12.98; (+) HRESIMS m/z 449.2510 [M+H]⁺ (calcd for C₂₅H₃₇O₇ 449.2534).

### Δ⁹-Tetrahydrocannabinol-1-O-β-D-Glucopyranoside (9-THCOG) (2a):

¹H NMR (400 MHz, MeOD) δ 6.56 (s, 1H), 6.41 (s, 1H), 6.29 (s, 1H), 4.96 (d, J=7.4 Hz, 1H), 4.03−3.86 (m, 1H), 3.76 (dd, J=12.0, 5.1 Hz, 1H), 3.54 (dd, J=13.6, 6.0 Hz, 1H), 3.51−3.41 (m, 3H), 2.51 (t, J=7.6 Hz, 2H), 2.24−2.10 (m, 2H), 2.07−1.80 (m, 1H), 1.67 (s, 3H), 1.66−1.55 (m, 4H), 1.51−1.43 (m, 1H), 1.41 (s, 3H), 1.39−1.27 (m, 4H), 1.08 (s, 3H), 0.93 (t, J=6.9 Hz, 3H); ¹³C NMR (100 MHz, MeOD) δ 156.30, 154.08, 142.23, 132.37, 125.51, 111.61, 111.04, 106.08, 100.35, 77.07, 76.82, 76.77, 73.82, 70.16, 61.30, 46.17, 35.43, 33.75, 31.22, 30.87, 30.63, 26.57, 24.88, 22.19, 22.16, 18.00, 13.03; (+) HRESIMS m/z 477.2827 [M+H]⁺ (calcd for C₂₇H₄₁O₇ 477.2847).

### Δ⁹-Tetrahydrocannabinol-2-C-β-D-Glucopyranoside (9-THCCG) (2b):

¹H NMR (400 MHz, MeOD) δ 6.39 (s, 1H), 6.06 (s, 1H), 4.54 (d, J=9.7 Hz, 1H), 3.80 (dd, J=12.2, 2.2 Hz, 1H), 3.74 (dd, J=12.2, 4.4 Hz, 1H), 3.61 (t, J=9.3 Hz, 1H), 3.46 (t, J=9.4 Hz, 1H), 3.38 (d, J=8.9 Hz, 1H), 3.36−3.29 (m, 1H), 3.14−3.03 (m, 1H), 2.77−2.68 (m, 1H), 2.32−2.24 (m, 1H), 2.13−2.00 (m, 2H), 1.89−1.80 (m, 1H), 1.56 (s, 3H), 1.54−1.49 (m, 1H), 1.48−1.38 (m, 2H), 1.33−1.21 (m, 8H), 0.92 (s, 3H), 0.82 (t, J=6.9 Hz, 3H); ¹³C NMR (100 MHz, MeOD) δ 154.98, 154.16, 141.11, 132.29 (2xC), 124.71, 110.66, 109.81, 81.28, 79.16, 78.31, 76.67, 72.38, 69.76, 60.71, 46.16, 33.88, 32.89, 31.48, 30.92, 30.37, 26.60, 24.96, 22.23, 22.06, 18.11, 13.05; (+) HRESIMS m/z 477.2821 [M+H]⁺ (calcd for C₂₇H₄₁O₇ 477.2847).

### Δ⁸-Tetrahydrocannabinol-1-O-β-D-Glucopyranoside (8-THCOG) (3a):

¹H NMR (400 MHz, MeOD) δ 6.55 (d, J=1.1 Hz, 1H), 6.30 (d, J=1.1 Hz, 1H), 5.45 (d, J=2.8 Hz, 1H), 4.92 (d, J=7.2 Hz, 1H, anomeric proton), 4.03−3.87 (m, 1H), 3.75 (dd, J=12.0, 5.0 Hz, 1H), 3.56−3.40 (m, 3H), 3.36−3.28 (m, 1H), 2.86 (td, J=11.1, 4.7 Hz, 1H), 2.51 (t, J=7.7 Hz, 2H), 2.25−2.10 (m, 1H), 1.94−1.79 (m, 1H), 1.80−1.64 (m, 5H), 1.66−1.53 (m, 2H), 1.48−1.27 (m, 8H), 1.08 (s, 3H), 0.94 (t, J=7.0 Hz, 3H); ¹³C NMR (100 MHz, MeOD) δ 156.82, 154.06, 142.25, 134.57, 118.99, 112.90, 111.10, 106.35, 100.43 (anomeric carbon), 77.15, 76.76, 76.20, 73.82, 70.13, 61.27, 45.50, 36.61, 35.42, 31.66, 31.24, 30.62, 27.60, 26.61, 22.33, 22.18, 17.26, 13.00; (+) HRESIMS m/z 477.2819 [M+H]⁺ (calcd for C₂₇H₄₁O₇ 477.2847).

### Δ⁸-Tetrahydrocannabinol-2-C-β-D-Glucopyranoside (8-THCCG) (3b):

¹H NMR (400 MHz, MeOD) δ 6.06 (s, 1H), 5.31 (s, 1H), 4.53 (d, J=9.7 Hz, 1H), 3.76 (ddd, J=16.6, 12.2, 3.4 Hz, 2H), 3.60 (t, J=9.3 Hz, 1H), 3.45 (t, J=9.3 Hz, 1H), 3.37 (d, J=8.9 Hz, 1H), 3.33 (dd, J=5.0, 2.8 Hz, 1H), 3.32−3.26 (m, 1H), 2.77−2.67 (m, 1H), 2.56 (td, J=11.0, 4.6 Hz, 1H), 2.33−2.23 (m, 1H), 2.04 (d, J=16.2 Hz, 1H), 1.72 (t, J=14.1 Hz, 1H), 1.63 (dd, J=11.2, 7.3 Hz, 1H), 1.59 (d, J=9.3 Hz, 3H), 1.56 (s, 1H), 1.51−1.39 (m, 2H), 1.30−1.21 (m, 7H), 0.94 (s, 3H), 0.82 (t, J=6.9 Hz, 3H); ¹³C NMR (100 MHz, MeOD) δ 155.56, 154.14, 141.15, 134.55, 118.94, 113.78, 112.09, 109.80, 81.31, 79.16, 78.34, 76.13, 72.38, 69.77, 60.73, 45.48, 35.89, 32.90, 31.92, 31.49, 30.37, 27.67, 26.62, 22.34, 22.21, 17.39, 13.02; (+) HRESIMS m/z 477.2823 [M+H]⁺ (calcd for C₂₇H₄₁O₇ 477.2847).

### Cannabinol-1-O-β-D-Glucopyranoside (CBNOG) (4a):

¹H NMR (400 MHz, MeOD) δ 8.37 (s, 1H), 7.04 (d, J=7.9 Hz, 1H), 6.95 (dd, J=7.9, 1.0 Hz, 1H), 6.64 (d, J=1.3 Hz, 1H), 6.36 (d, J=1.4 Hz, 1H), 5.06 (d, J=7.7 Hz, 1H), 3.80 (dd, J=12.1, 2.1 Hz, 1H), 3.62 (dd, J=12.1, 5.4 Hz, 1H), 3.57−3.50 (m, 1H), 3.43 (t, J=6.1 Hz, 1H), 3.41−3.37 (m, 1H), 3.37−3.29 (m, 1H), 2.52−2.40 (m, 2H), 2.26 (s, 3H), 1.58−1.53 (m, 2H), 1.45 (s, 3H), 1.42 (s, 3H), 1.30−1.23 (m, 4H), 0.82 (t, J=7.0 Hz, 3H); ¹³C NMR (100 MHz, MeOD) δ 154.80, 154.20, 144.22, 136.84, 136.35, 127.72, 127.24, 127.19, 121.87, 111.66, 110.84, 108.37, 100.45, 77.33, 76.90, 76.86, 73.83, 70.06, 61.19, 35.57, 31.26, 30.49, 25.96, 25.93, 22.21, 20.17, 13.04; (+) HRESIMS m/z 473.2517 [M+H]⁺ (calcd for C₂₇H₃₇O₇ 473. 2534).

### Cannabinol-2-C-β-D-Glucopyranoside (CBNCG) (4b):

¹H NMR (400 MHz, MeOD) δ 8.30 (s, 1H), 7.05 (d, J=7.9 Hz, 1H), 6.94 (dd, J=7.9, 1.0 Hz, 1H), 6.24 (s, 1H), 4.63 (d, J=9.7 Hz, 1H, anomeric proton), 3.88−3.68 (m, 3H), 3.52 (t, J=9.4 Hz, 1H), 3.42 (d, J=9.0 Hz, 1H), 3.39−3.33 (m, 1H), 2.81−2.71 (m, 1H), 2.45−2.29 (m, 1H), 2.24 (s, 3H), 1.56−1.48 (m, 2H), 1.47 (s, 3H), 1.41 (s, 3H), 1.31−1.26 (m, 4H), 0.84 (t, J=7.0 Hz, 3H); ¹³C NMR (100 MHz, MeOD) δ 154.46, 153.82, 143.01, 136.75 (2xC), 136.07, 127.69, 127.22, 126.99, 121.95, 110.29, 110.13, 81.29 (anomeric carbon), 79.05, 78.30, 76.89, 72.24, 69.73, 60.66, 32.97, 31.49, 30.30, 26.16, 26.10, 22.21, 20.17, 13.01; (+) HRESIMS m/z 473.2514 [M+H]⁺ (calcd for C₂₇H₃₇O₇473.2534).

### Example 4 Measurement of Cytotoxicity

Cell viability assay: The cell viability was determined by the standard MTT assay method. Briefly, Panc-1, HCT-116, A549, PC3, MIAPaca-2, HT-29, MDA-MB-231, MCF7 cells, and fR2cells were seeded in 96 well tissue culture plates (Nunc) at a density of 4×10³ cells per well and treated with varying concentrations of the test compounds in triplicates. DMSO was taken as a vehicle, and its final concentration was maintained at 0.2% in the culture medium. Doxorubicin was employed as a positive control. After 44 h of incubation, MTT dye solution was added into the medium, and cells were incubated for another 4 h at 37° C. in 5% CO₂. The amount of colored formazan derivatives formed was measured by taking optical density (OD) using a microplate reader (TECAN, Infinite M200 Pro) at 570 nm, and the percentage of cell viability was calculated. The IC₅₀ values were calculated using GraphPad Prism software (Version 5.0).

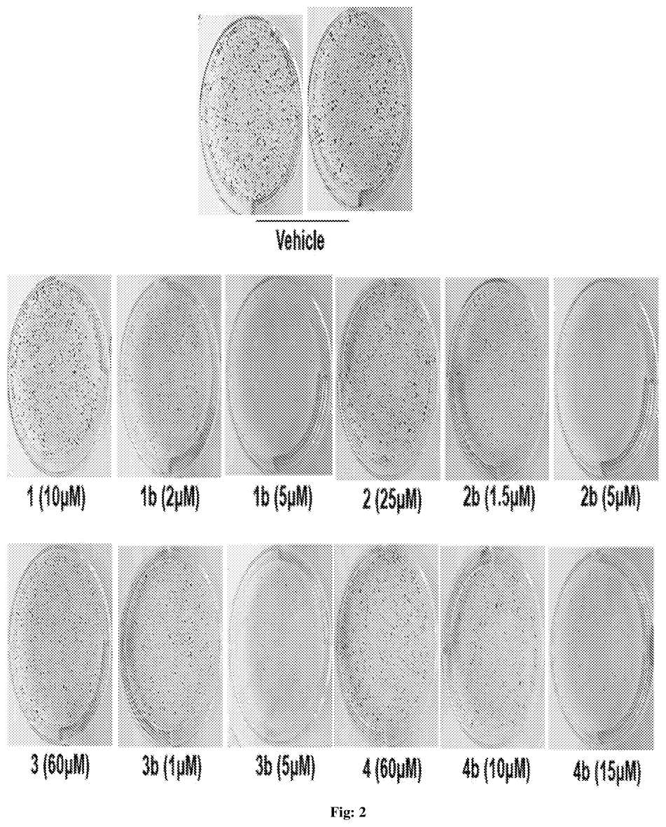

### Example 5 Measurement of Anti-Proliferative Activities (FIG. 2)

Colony formation assay. MIAPaca-2 cells were seeded onto 6 well plates with a seeding density of 1000 cells/well. The cells after attachment were treated with 2 (2a, 2b), 3 (3a, 3b), 4 (4a, 4b) along with DMSO as a vehicle control and incubated for 5-6 days. After incubation, the plates were washed with ice-cold PBS, and the cells were fixed with methanol for 5 min. After fixing. the cells were stained with 0.2% crystal violet stain for 1 h. The cells were then washed with distilled water to remove the stain and were observed under an inverted microscope at 20× magnification.

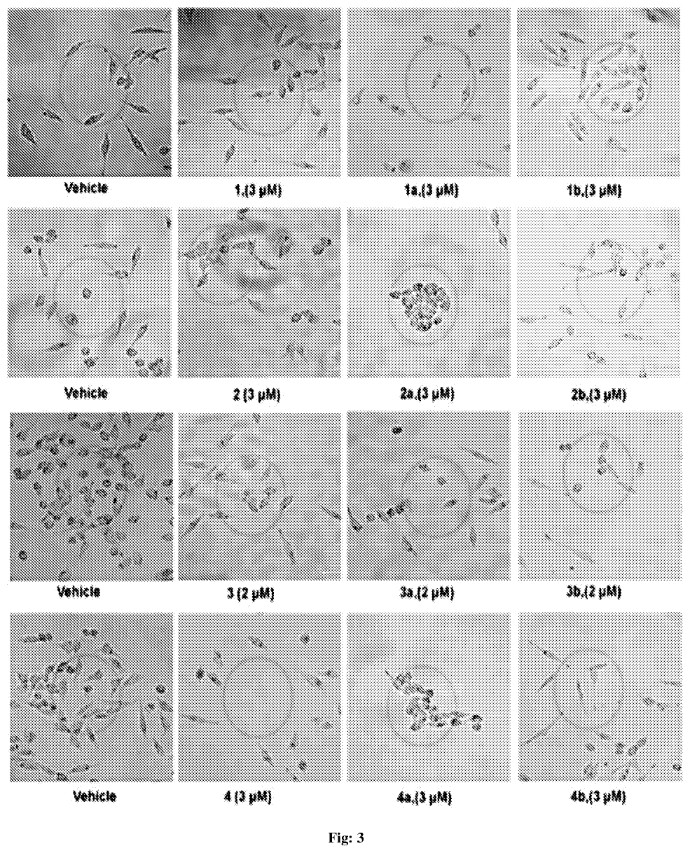

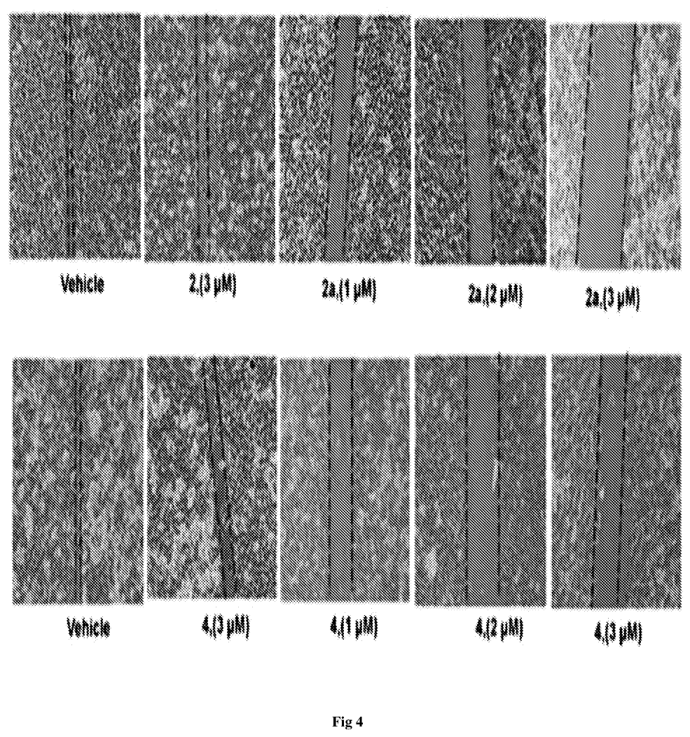

### Assessment of Anti-Migratory Properties (FIGS. 3 and 4)

Cell scatter assay MIAPaCa-2 cells were seeded onto a 12 well plate at a seeding density of 500 cells/well and kept for 4-5 days, and there was the formation of scatter colonies since the cell line itself is a metastatic cell line. Accordingly, they were treated with 1 μM, 2 μM & 3 μM of compound 2, 2a, 2b, 3, 3a, 4, 4a & 4b for 36 h, and upon termination, they were washed with PBS and observed under an inverted microscope at 20× magnification (FIG. 3).

Wound healing assay. MIAPaCa-2 cells (1×10⁶) were seeded onto 6 well plates and grown up to more than 90% confluency, and wounds were created with sterile pipette tip (20-200 μL), and the media was replaced with serum-free media. Subsequently, the cells were treated with vehicle (DMSO), compounds 3 μM of 2 and 4, and their derivatives, i.e., 1, 2 and 3 μM of 2a, 2b, 4a, 4b for 36 h. Upon termination, the cells were washed with PBS, and images were captured under a microscope at 20× magnification (FIG. 4).

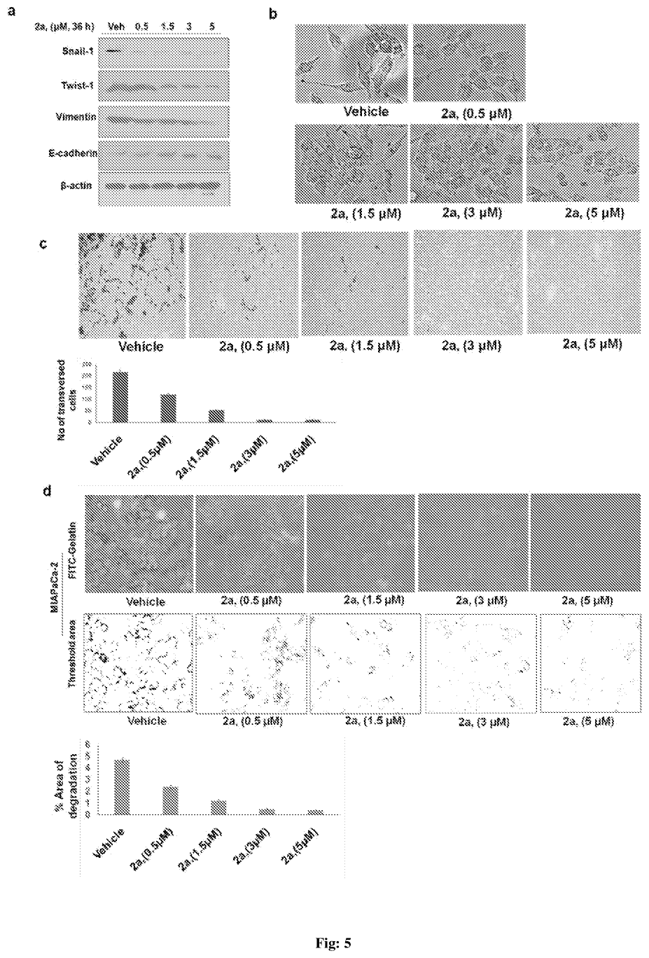

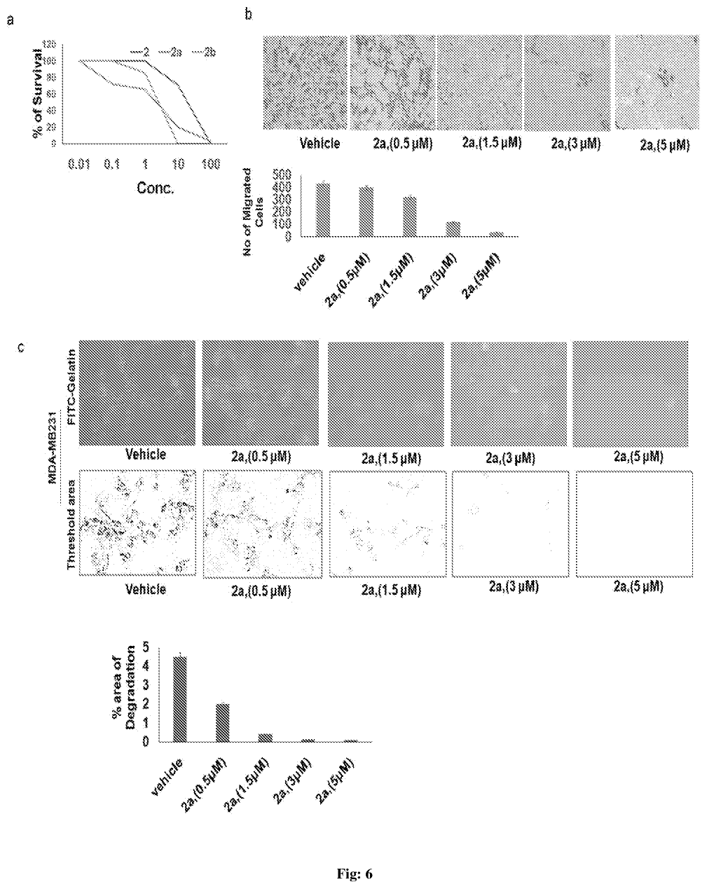

### Example 6 Assessment of Anti-Invasive Properties and Suppression of EMT Markers (FIGS. 5 and 6)

Matrigel invasion assay: MIAPaCa-2 and MDA-MB-231 cells (1×10⁶) were seeded onto matrigel coated inserts and treated with compound 2a (0.5, 1.5, 3 & 5 μM) for 36 h in 5% CO₂ incubator at 37° C. The upper chamber is filled with serum-free media, and the lower half consists of 10% FBS supplemented media, thus creating a gradient of chemoattractant that facilitates the migration of cells. Upon termination, the inserts were washed with PBS, and the non-migratory cells were removed from the upper chamber by using a cotton plug. Further, the cells were fixed with methanol and stained later with 0.2% crystal violet. After drying, the inserts were observed under an inverted microscope at 20× magnification, and images were taken.

Western Blot analysis. Briefly, MIAPaCa-2 cells were seeded in a 60 mm petri dish and maintained up to 70% confluency; afterward, the cells were treated with compound 2a (0.5, 1.5, 3 & 5 μM) for 36 h. After treatment, the cells were harvested washed thrice with ice-cold PBS and were subjected to protein lysis with lysis buffer (HEPES 1 mM/L, KCl 60 mM/L, NP-40 0.3%, EDTA 1 mM/L, DTT 1 mM/L, sodium orthovanadate 1 mM/L, PMSF 0.1 mM/L and cocktail protease inhibitor). The lysis product was centrifuged at 12000 rpm for 15 min at 4° C. to remove the cellular debris, and the supernatants were collected. Total protein was estimated with the help of the Bradford method. An equal amount of protein was subjected to SDS-PAGE, and consequently, after the protein was separated on SDS-PAGE based on their molecular weight, they were transferred onto the PVDF membrane. The membrane was blocked with 5% BSA and incubated in primary antibody prepared in 5% BSA (dilution ranging from 1:1000-1:2000) for 2-3 h. The membrane was then washed with TBST buffer for 30 min and incubated with species-specific secondary antibodies tagged with horseradish peroxide. After washing with TBST, the protein expression was quantified by adding a chemiluminescent substrate on the membrane, and the signal obtained was captured in Biomax light x-ray films.

### Example 7 Assessment of In-Situ Gelatin Degradation Assay to Examine the Formation of Migratory Structures (FIGS. 5 and 6)

Matrix gelatin degradation assay. In-situ gelatin degradation assay is performed to detect the formation of migratory structures (invadopodia & filopodia). Briefly, glass coverslips were coated with gelatin tagged with FITC, and the coated coverslips were dipped into 70% ethanol and immersed into a 6 well plate filled with RPMI and the coverslips were kept in a CO₂ incubator for 1-2 h for preconditioning. Later on, both MIAPaCa-2 and MDA-MB-231 cells were seeded onto the coverslips and kept overnight for attachment. Treatment of the compound 2a (0.5, 1.5, 3 & 5 μM) was given on the next day and continued for 36 h. Upon termination, the coverslips were washed with ice-cold PBS, and then it was fixed in 4% paraformaldehyde for 15 min and then washed with PBS. Finally, the coverslips were mounted using mounting media consist of glycerol, and the edges were sealed with nail polish. After drying, the slides were observed under a fluorescence microscope to assess a degraded area's presence. The fluorescent images were captured, and the images were further processed in Image J software (Version 1.50i) to quantify the degraded area in each field.

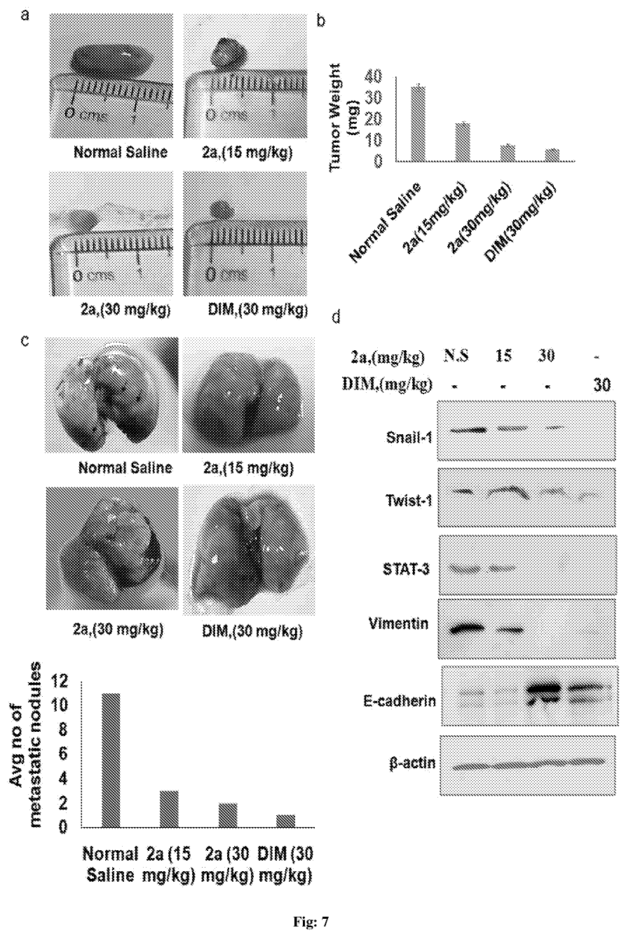

### Example 8 In-Vivo Studies (FIG. 7)

In vivo anti-metastatic and anti-tumor efficacy studies. 4T1 mouse mammary carcinoma cells is a highly aggressive cell line that forms tumors when grafted in Balb/c mice. Hence, to study the anti-tumor/anti-metastatic activity of compound 2a, healthy Balb/c mice of weight ranging from 18-25 g were taken. All the in vivo experimental protocols were approved by the Institutional Animal Ethics Committee, CPCSEA, of the Indian Institute of Integrative Medicine, Jammu. The animals were maintained at 22° C. with a 12 h light-dark cycle and free access to feed and water inside the institutional animal house. Proper care was taken to maintain them in a healthy condition and to avoid any risk of possible pathogenic contaminations. For subcutaneous implantation of 4T1 cells, the Balb/c mice were distributed into four groups bearing five mice in each group. For tumor induction, 1.5 million 4T1 cells were suspended in serum-free media and injected into the mammary pad of the animal and kept in the animal house for tumor induction. After the tumor reached an adequate size (30-150 mm³) the mice in the allotted groups were treated with vehicle (normal saline), 15 and 30 mg/kg 2a & 30 mg/kg DIM (positive control) for every alternate day and was continued for two weeks. After the treatment, the mice were sacrificed by cervical dislocation, and the tumor was dissected out from the mammary pad, the tumor volume was measured, and images were taken. Subsequently, to examine the anti-metastatic property of the compound 2a, the chest cavity was dissected to remove the lungs, and after washing with PBS, the metastatic nodules in the lung due to 4T1 migration were checked and subsequently counted.

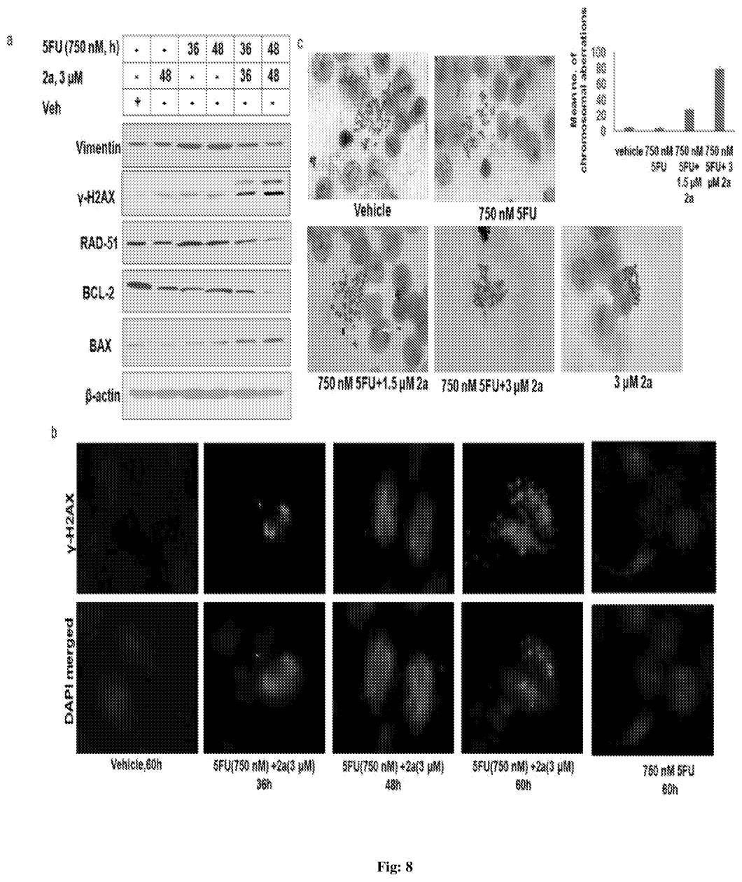

### Example 9 Drug Potentiation Assay1 (FIG. 8)

Assessment of DNA damage by combinatorial treatment of 5FU and Compound 2a. An immunofluorescence study was performed to assess the expression of γH2AX, which tends to form a complex with damaged DNA resulting in the formation of a beads-like structure when tagged with a fluorescent labelled antibody. Briefly, HCT-116 cells were seeded in 8 well chamber slide and treated with vehicle, 750 nM 5FU+3 μM 2a for 36, 48, and 60 h and 750 nM 5FU for 60 h. Following termination of drug treatment, the cells were washed with ice-cold PBS and fixed with 4% paraformaldehyde for 10 min. Subsequently, permeabilization was done by incubating the cells with 0.1% Triton X-100. Before incubating with primary antibody (anti-γH2AX mouse monoclonal antibody), the cells were blocked with 5% BSA for 1-2 h; consequently, the cells were incubated with primary antibody (1:200) for overnight at 4° C. Subsequently, the cells were washed with ice-cold PBS for five times and further incubated with secondary anti-mouse antibody (1:500) for 30 min. The slides, after thorough washing with PBS, were mounted in DAPI containing mounting media. The images were captured in Floid imaging station at 20× optical magnification, and digital zooming was done up to 100 μM.

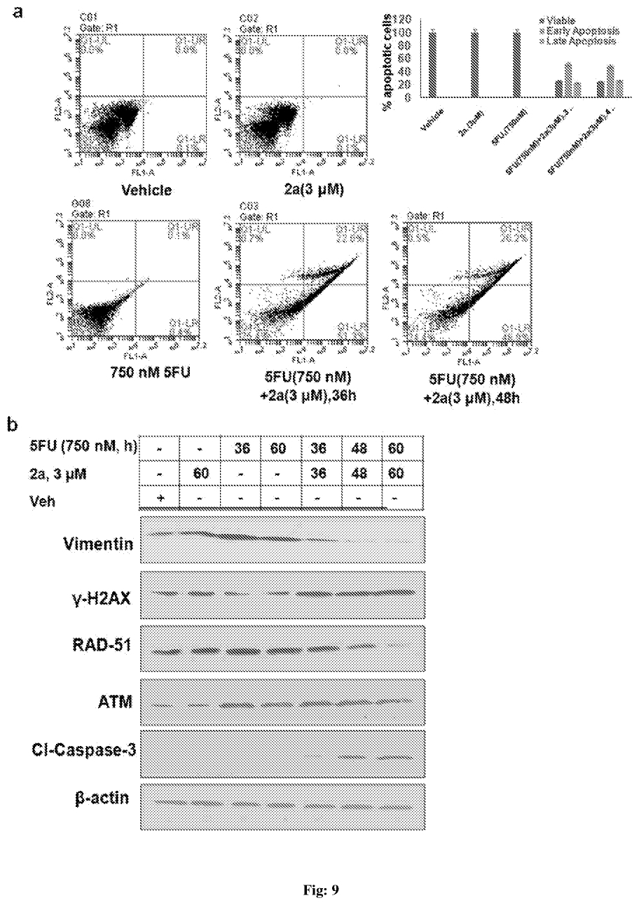

### Example 10 Drug Potentiation Assay 2 (FIG. 9)

### Assessment of Apoptotic Stages in the Presence of 5FU and Compound 2a

1×10⁶ HCT-116 cells were seeded in 60 mm Petri dish and treated with vehicle, 3 μM 2a, 750 nM 5FU and 750 nM 5FU+3 μM 2a for indicated time points. After treatment termination, the cells were carefully trypsinized and the palette obtained were resuspended in 600 μL of binding buffer (10 mM HEPES [N-2-hydroxyethylpiperazine-N-2-ethanesulfonicacid], 140 mM NaCl, and 2.5 mM CaCl₂, pH 7.4). Subsequently, the cells were stained with 6 μL of AnnexinV-FITC and 10 μL of propidium iodide for 20 min at room temperature in the dark. The samples were immediately analyzed by FACS (BD Accuri C6), and the scatter plot was obtained by BD Accuri C6 software.

### Anti-Proliferative Property of C— and O-Glycosidic Modifications of Phytocannabinoids

The C— and O-glycosides of Cannabinoids possess anti-proliferative properties and majority of the compounds display mild to high toxicity against the panel of pancreatic, colon, lung, prostrate, breast cancer cell lines and a normal epithelial cell line as depicted in Table-1.

TABLE 1
Cytotoxicity (IC₅₀) of glycosidic modification of Phytocannabinoids

| Compounds | Panc-1 | MIAPaCa-2 | HCT-116 | HT-29 | A549 | PC3 | MCF-7 | fR2 |
|---|---|---|---|---|---|---|---|---|
| 1 | 9.28 | 23.17 | 10.66 | 46.17 | 39.37 | 88.2 | >100 | NA |
| 1a | 21.43 | >100 | 32.91 | >100 | 53.07 | >100 | 90.60 | NA |
| 1b | 4.12 | 9.2 | 3.91 | 40.18 | 35.7 | 24.046 | >100 | NA |
| 2 | 21.12 | 4.47 | 18.97 | >100 | 11.72 | >100 | 43.02 | NA |
| 2a | 3 | 1.42 | 2.1 | 3.162 | 1.44 | 7.17 | 15.39 | >100 |
| 2b | 3.16 | 1.59 | 43.18 | 10.75 | 1.32 | 28 | 7.40 | NA |
| 3 | 50.88 | 2.46 | 27.27 | >100 | 11.56 | 28 | 26.65 | NA |
| 3a | 3.16 | 0.742 | 30.19 | 10.74 | 2.15 | 38 | 34.87 | >100 |
| 3b | 3 | 0.901 | 20.52 | 8.39 | 2.39 | 51.2 | 19.53 | >100 |
| 4 | 11.83 | 1.63 | 47.11 | 20.35 | 2.13 | 28 | 50.64 | NA |
| 4a | 3.16 | 2.58 | 2.98 | 3.53 | 3.01 | 51.4 | 7.74 | >100 |
| 4b | 13.92 | 8.31 | 3 | 11.45 | 22.55 | 39 | 43.12 | NA |

Specifically, the novel C-glycosidic derivatives 1b, 2b, 3b and 4b displayed consistent anti-proliferative properties against diverse cancer cells. The compounds 1b and 2b showed >2-fold (9.28 μM vs 4.12 μM) and 6.6-fold (21.12 μM vs 3.16 μM) activity in the pancreatic cancer cells, respectively as compared to its respective parent compounds (1&2). Similarly, the compound 3b is found 16-fold (50.88 μM vs 3.0 μM) more potent than its parent compound 3. Moreover, the compounds 2b (more than 10-fold; >100 μM vs 10.75 μM) and 3b (more than 4.8-fold; 11.56 μM vs 2.29 μM) found to possess strong anti-proliferative activity as compared to parent compounds 2 and 3 against human colorectal adenocarcinoma cell line (HT-29) and lung adenocarcinoma cells (A549), respectively. Interestingly, compound 4b showed significant anti-proliferative activity (>15-fold: 47.11 μM vs 3.0 μM) against colorectal adenocarcinoma cells (HCT-116) compared to its parent compound 4. The anti-proliferative properties of glycosidic derivatives were further analysed by colony formation assay in pancreatic cancer cells. The novel C-glycosidic derivatives of compounds 1, 2, 3 and 4 viz 1b, 2b, 3b & 4b display anti-proliferative property (FIG. 2). Panc-1cells were seeded onto 6 well plates at seeding density 1000 cells/well and treated with indicated concentration of 1b, 2b, 3b and 4b derivatives along with their parent compounds and DMSO as vehicle for 5 days. Upon termination the cells were stained with crystal violet and images of field were photographed. The results demonstrated strong anti-proliferative potential of novel C-glycosidic compounds 1b, 2b, 3b and 4b as compared to their corresponding parent compounds (FIG. 2).

### Screening of Anti-Metastatic Properties of C and O-Glycosidic Modifications of Phytocannabinoids

The data obtained from the above anti-proliferative assay demonstrated that the derivatives of compounds 1, 2, 3 and 4 showed significant cytotoxic activities compared to their parent compounds in a wide array of cancer cell lines. Rationally, these compounds were further tested for their anti-metastatic activities. The C— and O-glycosidic derivatives of 2 (2a, 2b) and 4 (4a, 4b) displayed typical clumping of scatter cells compared to vehicle as well as parent molecule 2 and 4 in cell scattering assay. The derivatives of compound 3 (3a, 3b) although showed significant anti-proliferative effect but did not prevent cell scattering (FIG. 3). Hence, to understand more comprehensively about the anti-migratory property of derivatives of compounds 2 and 4, wound healing assay was performed (FIG. 4). From the above results, it has been confirmed that although compound 2a is known but its novel biological activities (anti-metastatic properties) are not reported elsewhere. The data implied that compound 2a showed the most prominent anti-migratory properties as compared to compounds 2, 2b, 4 and its derivative 4a as well as 4b.

### Compound 2a Inhibits the EMT Processes and Hampers Pancreatic Cancer Cells Invasion

The results obtained so far suggested that compound 2a possess the most potential anti-invasive characteristics in scatter and wound healing assays (FIGS. 5 & 6). Now this particular derivative was further analysed for its ability to inhibit the molecular signatures of EMT. To this end, MIAPaCa-2 cells were treated with 0.5 μM, 1.5 μM, 3 μM and 5 μM of compound 2a for 36 h and were subjected to immunoblotting to study the expression of Snail-1, Twist-1, Vimentin and E-cadherin. The results showed that with increasing concentration of compound 2a, there was a significant down modulation of Snail-1, Twist-1 and Vimentin and E-cadherin which is a canonical marker for epithelial cells got upregulated from 1.5 to 5 μM (FIG. 5). The MIAPaCa-2 cells treated with indicated concentration of compound 2a when observed under phase contrast microscope showed gradual loss of mesenchymal (fibroblastic) morphology starting from 0.5 μM to 5 μM (FIG. 5b). To analyse more deeply the ability of this compound to affect migration/metastasis, MIAPaCa-2 cells were seeded onto Matrigel coated inserts and were treated with 0.5 μM, 1.5 μM, 3 μM and 5 μM of compound 2a. The results obtained clearly depicted the inhibition of cellular invasion periodically from 0.5 μM concentration of compound 2a (FIG. 5c). Formation of invadopodia and filopodia (migratory structures) constitutes the fundamental property of cells undergoing migration. In order to study these structures in-situ gelatin degradation assay was performed. 1×10⁶ cells (MIAPaCa-2) were seeded onto gelatin-FITC coated slides kept in 6 well plate and treated subsequently with indicated concentrations of compound 2a for 36 h. There was a significant loss of gelatin degraded area starting from 0.5 μM onwards indicating the loss of migratory structures hence potential inhibition of migration/invasion.

### Compound 2a Abrogates Invasion and Metastasis of Highly Aggressive Breast Cancer Cells

The compound 2a showed potential anti-invasive property against a highly metastatic cancer cell of pancreatic origin (MIAPaCa-2). Now to study its anti-migratory and anti-metastatic role in another highly metastatic cancer cells of breast origin (MDA-MB 231) the IC₅₀ value was determined by performing MTT assay (FIG. 6a). Interestingly, the compound 2a showed much higher IC₅₀ (15.39) value in case of MCF-7, a breast cancer cell of epithelial origin compared to MDA-MB 23 (IC₅₀ 1.42) an aggressive breast cancer cell line of mesenchymal origin. Hence, to analyse the anti-migratory role of compound 2a, MDA-MB231 cells were subjected to Matrigel coated invasion chamber assay system. The results obtained clearly described the periodical loss of migration of MDA-MB231 cells from concentration 0.5 μM to 5 μM (FIG. 6b). Further, to understand the inhibitory role of this compound in forming migratory structures (invadopodia & filopodia), gelatin degradation assay was performed and significant decrease of gelatin degradation from concentration 1.5 μM to 5 μM (FIG. 6c) was observed.

### Compound 2a Stalls Tumor Growth and Metastasis in 4T1 Mouse Metastatic Model

The anti-proliferative as well as anti-metastatic role of compound 2a was studied in 4T1 allograft breast cancer model. Briefly, 1.5×10⁶ 4T1 mammary breast cancer cells were injected in mouse mammary pad and kept undisturbed until the formation of tumor. Subsequently, the mice were treated with 15 and 30 mg/kg of compound 2a along with a positive control 30 mg/kg di-indoyl methane (DIM) molecule and normal saline as vehicle. The drugs were administered intraperitonially for each alternate day for 2 weeks. The animals were sacrificed and tumours were harvested from the mammary pad and tumor weight was measured (FIG. 7a & b). Moreover, the lungs were also removed and photographed to study the presence of metastatic patches. After analysing the tumor parameter viz tumor weight which was drastically reduced in compound 2a treated cohorts as compared to normal saline suggesting the anti-proliferative property of A9-tetrahydrocannabinol derivative (2a) in mouse breast cancer model (FIG. 8a). The 4T1 tumor once form transversed to lung spontaneously and form white patches surrounding the lungs which was observed in normal saline cohort but in treated groups (15 & 30 mg/kg) the white patches were not observed suggesting the potential anti-metastatic property of the 2a (FIG. 7c). Furthermore, the tumors were subjected to tissue lysis and immunoblot analysis was performed to study the expression of Snail-1. Twist-1, STAT-3, Vimentin and E-cadherin. The results obtained demonstrated that the expression of Snail-1. STAT-3 and Vimentin decreases significantly at 15 and 30 mg/kg concentration as well as in positive control (DIM, 30 mg/kg). The expression of Twist-1 was curbed at 30 mg/kg concentration of compound 2a and the epithelial marker, E-cadherin level increased considerably at 30 mg/kg concentration (FIG. 8d). Cumulatively, the results suggested that compound 2a is a potential anti-proliferative molecule against a range of cancers (colon, pancreatic and breast) and it possess a very prominent anti-invasive and anti-metastatic property as studied in pancreatic and breast cancer model.

### Compound 2a in Combination Elevates the DNA Damaging Potential of 5-Fluorouracil in Colon Cancer

The effect of the compound 2a in combination with DNA damaging drug 5-Fluorouracil in colon adenocarcinoma cells was studied. HCT-116 cells treated with 5FU in combination with compound 2a for 36 and 48 h displayed enhanced expression of γH2AX protein indicating the potentiation of DNA damaging effect of 5FU. Consequently, the major repair factor RAD-51 got diminished as compared to 5FU alone treated lane. The immunoblot results also depicted the downmodulation of cellular survival factor BCL-2 and subsequent upregulation of proapoptotic protein Bax (FIG. 8a). Similarly, the immunocytochemistry results also showed increased expression of γH2AX in lanes treated with 5FU in combination with compound 2a indicating elevation of DNA damage (FIG. 8b). Moreover, the effect of the combinatorial treatment on chromosomal stability in HCT-116 cells by chromosome isolation and staining was studied. The results depicted that the compound 2a in combination with 5FU significantly increases chromosomal abbreations as compared to vehicle/750 nM 5FU/3 μM 2a (FIG. 8c).

### Compound 2a in Combination Increases the Apoptosis Potential of 5FU

The DNA damaging effect of compound 2a in combination with subtoxic doses of 5FU was further analyzed by Annexin/PI staining to check the activation of programmed cell death. The data obtained clearly demonstrated the induction of late phase of apoptosis (Annexin⁺/PI⁺) in lane treated with 5FU in combination with compound 2a (FIG. 10a). Furthermore, the compound 2a in association with 5FU can able to incite caspase-3 cleavage at 48 and 60 h time points, hence indicating stimulation of committed apoptotic stages. Cumulatively, compound 2a elevated the DNA damaging effect and eventually leads to programmed cell death in combination with subtoxic doses of 5FU (FIG. 9b).

It has been found that the O-glycosidic modification of Δ⁹-tetrahydrocannabinol (2a) drastically augmented its parent natural product's anti-proliferative activities in a wide range of cancer cell lines. The 2a significantly abrogated the migration and invasion of highly metastatic cancer cells (MIAPaCa-2 & MDA-MB-231) of pancreatic and breast cancer origin. Moreover, compound 2a diminishes major EMT (Epithelial to Mesenchymal Transition) related transcription factors viz. Snail-1 and Twist-1 were conferring vital role in cancer metastasis.

Additionally, compound 2a severely impeded the aggressive 4T1 cells transplanted mice mammary pad tumor volume along with its devastating metastatic spread. Moreover, this compound combined with 5-Fluorouracil (5FU), a widely used drug in clinical practice, nullifies the drug-induced EMT/survival responses; hence, potentiating the DNA damaging effects of 5FU, leading to activation of programmed cell death in colon cancer cells. In a nutshell, the present invention describes the novel antitumor/anti-metastatic activities of compound 2a in diverse cancer cells rendering its integrative therapeutic approaches in breast and pancreatic origin cancer models.

### ADVANTAGES OF THE INVENTION

The present invention describes the fractionation method as a tool for separation of a distinct class of Cannabinoids by solid-phase extraction using HP-20 resins, which is not reported yet.
Herein one of the fractions containing a mixture of 1-5 distinct Cannabinoids compound that were chemically transformed into several distinct synthetic C— & O-glycosylated Cannabinoids such as 1a, 1b, 2a, 2b, 3a, 3b, 4a and 4b in one step.
The compounds 1b and 2b showed >2-fold (9.28 μM vs 4.12 μM) and 6.6-fold (21.12 μM vs 3.16 μM), respectively activity as compared to their respective parent compounds (1&2).
Similarly, the compound 3b was found to be 16-fold (50.88 μM vs 3.0 μM) more potent than its parent compound 3. Moreover, the novel compounds 2b (more than 10-fold; >100 μM vs 10.75 μM) and 3b (more than 4.8-fold; 11.56 μM vs 2.29 μM) were found to possess strong anti-proliferative property compared to their respective parent compounds 2 and 3 against lung adenocarcinoma cells (A549).
Interestingly, compound 4b showed significant anti-proliferative activity (>15-fold: 47.11 μM vs 3.0 μM) against colorectal adenocarcinoma cells (HCT-116) compared to its parent compound 4.
In search of anti-metastatic activities, it has been found that the most active molecule across multiple cell lines was compound 2a (A derivative of Δ9-tetrahydrocannabinol and its anti-metastatic property is novel); although most potent molecules in terms of cytotoxicity were 3a and 3b.
Further, this active compound 2a strongly abrogated migration, invasion, and FITC-gelatin matrix degradation ability of human pancreatic and breast cancer cells in the in vitro experimental setup, whereas it was found non-toxic towards normal human breast epithelial cells.
It was found that Compound 2a attenuated EMT cascades by diminishing the expression of prominent EMT markers, viz. Twist-1, Snail-1, Vimentin, and augmenting the level of epithelial marker E-cadherin.
Compound 2a was also found to be an effective inhibitor of tumor growth and metastasis in mouse mammary carcinoma model.
Compound 2a in combination with 5FU, augmented the DNA damaging effects of 5FU by escalating the expression of γH2AX and suppressing RAD-51 protein.
Additionally, compound 2a, in association with 5FU, was found to induce robust apoptosis in a mitochondrial pathway by activating ATM kinase and cleaving caspase-3.
The compound 2a can be used as a monotherapy against advanced forms of human Pancreatic/Breast cancer and also can potentiate the effect of 5FU in such metastatic diseases when used in combination.

1-10. (canceled)

11. A cannabinoid C— and O-glycoside compound having formula (A): 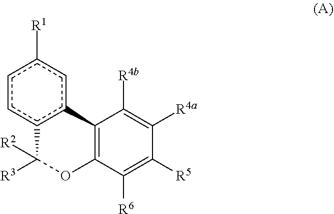
where:
R¹, R² and R³ are each independently selected from the group consisting of —H, —OH, alkyl, alkenyl, and alkynyl;
R^{4a} and R^{4b} are each independently selected from the group consisting of —H, O-glycoside, substituted O-glycoside, C-glycoside, substituted C-glycoside, —(CH₂)ₙ—O-glycoside, and —(CH₂)ₙ—C-glycoside; and
R⁵ and R⁶ are each independently selected from the group consisting of —H, halogen, —CN, —NO₂, —OH, -alkyl, —O-alkyl, and —COOH.

12. The cannabinoid C— and O-glycoside compound of claim 11, wherein the compound is selected from the group consisting of
Δ⁸-tetrahydrocannabivarin-1-O-β-D-glucopyranoside (8-THCVOG): 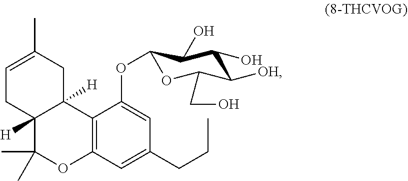
Δ⁸-tetrahydrocannabivarin-2-C-β-D-glucopyranoside (8-THCVCG): 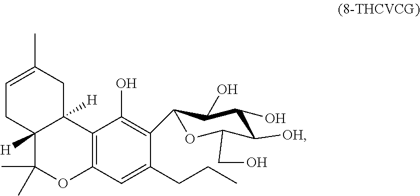
Δ⁹-tetrahydrocannabinol-1-O-β-D-glucopyranoside (9-THCOG): 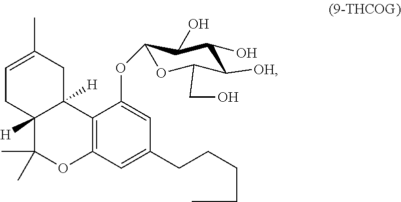
Δ⁹-tetrahydrocannabinol-2-C-β-D-glucopyranoside (9-THCCG): 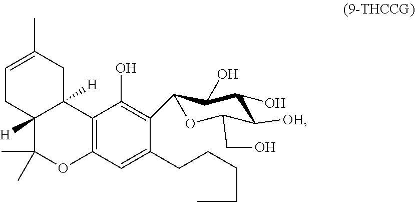
Δ⁸-tetrahydrocannabinol-1-O-β-D-glucopyranoside (8-THCOG): 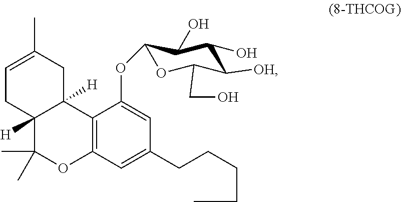
Δ⁸-tetrahydrocannabinol-2-C-β-D-glucopyranoside (8-THCCG): 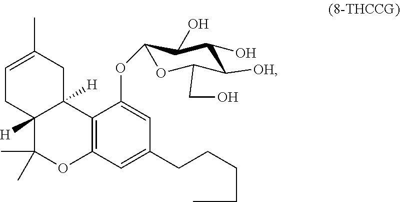
Cannabinol-1-O-β-D-glucopyranoside (CBNOG): 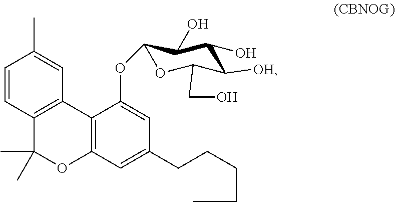
and
Cannabinol-2-C-β-D-glucopyranoside (CBNCG):

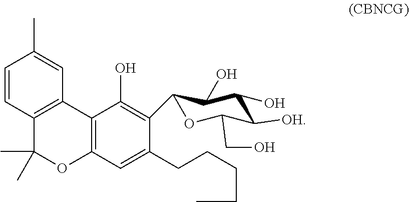

13. The cannabinoid C— and O-glycoside compound of claim 12, wherein the compound is selected from the group consisting of THCVCG, 9-THCCG, 8-THCCG, and CBNCG.

14. The cannabinoid C— and O-glycoside compound of claim 11, wherein the compounds possess anti-proliferative and anti-metastatic properties and effectively abrogates proliferation of different cancer cells in-vitro.

15. The cannabinoid C— and O-glycoside compound of claim 11, wherein the compound is Δ⁹-tetrahydrocannabinol-1-O-β-D-glucopyranoside (9-THCOG), and wherein the compound possesses anti-metastatic property and efficiently blocks migration and invasion of pancreatic cancer cells in vitro, suppresses murine mammary tumor growth and metastasis in vivo, and augments efficacy of anti-cancer drug 5FU in colorectal cancer.

16. A process for synthesizing the cannabinoid C— and O-glycoside compound of claim 11, the process comprising:
(a) extracting ground leaves of Cannabis sativa with hexane at room temperature to yield a crude extract;
(b) subjecting the crude extract to fractionation in an open column using HP-20 as a solid phase and a gradient solvent system with H2O—MeOH of 9:1, 8:2, 7:3, 6:4, 1:1, 4:6, 3:7, 2:8, 1:9 and 100% MeOH, resulting in cannabinoid enriched fractions 1 to 10.
(c) co-evaporating at least one of the cannabinoid enriched fractions 1 to 10 and a trichloroacetimidate glycoside donor of formula (I): 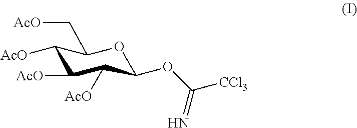
to obtain the cannabinoid C— and O-glycoside compound of formula (A).

17. The process of claim 16, wherein the at least one cannabinoid enriched fraction of (c) co-evaporated with trichloroacetimidate comprises fraction 5.

18. A pharmaceutical composition comprising the cannabinoid C— and O-glycoside compound of claim 11 and a pharmaceutically acceptable excipient.

19. The pharmaceutical composition of claim 18, wherein the pharmaceutically acceptable excipient is selected from the group consisting of calcium carbonate, sodium carbonate, lactose, calcium phosphate, sodium phosphate, oily suspensions, glycerol, propylene glycol, sorbitol, and sucrose.

20. A pharmaceutical composition comprising the cannabinoid C— and O-glycoside compound of claim 11, 5-fluorouracil, and a pharmaceutically acceptable excipient.

21. The pharmaceutical composition as claimed in claim 19, wherein the pharmaceutically acceptable excipient is selected from the group consisting of calcium carbonate, sodium carbonate, lactose, calcium phosphate, sodium phosphate, oily suspensions, glycerol, propylene glycol, sorbitol, and sucrose.
